(19) United States
(12) Patent Application Publication
(10) Pub. No.: US 2019/0307374 A1
(43) Pub. Date: Oct. 10, 2019

(54) DEVICES WITH SEPARATE SWEAT MANAGEMENT FOR STIMULATION AND SENSOR AREAS

(71) Applicant: University Of Cincinnati, Cincinnati, OH (US)

(72) Inventors: Jason Charles Heikenfeld, Cincinnati, OH (US); Zachary Cole Sonner, Elsmere, KY (US); Phillip Simmers, Powell, OH (US); Adam Hauke, Milford, OH (US)

(21) Appl. No.: 16/309,679

(22) PCT Filed: Jun. 27, 2017
(86) PCT No.: PCT/US2017/039421

§ 371 Date: Dec. 13, 2018

Related U.S. Application Data
(60) Provisional application No. 62/354,930, filed on Jun. 27, 2016.

(51) Int. Cl. A61B 5/145; A61B 5/00

(52) U.S. Cl. CPC A61B 5/14521; A61B 10/0064; A61B 5/4266

(57) ABSTRACT

A device (100) for sensing sweat on skin (12) includes a first analyte-specific sensor (120, 122) for sensing a first analyte in sweat; a sweat stimulating component (140); a waste collector (142, 144, 146, 148) beneath the sweat stimulating component (140); and an additional component being one or more of a sweat collector (130, 132, 134) or an additional analyte-specific sensor (124) for sensing an additional analyte in sweat that is different from the first analyte, the additional component being adjacent to and fluidically isolated from the waste collector (142, 144, 146, 148). A device (200) for sensing sweat on skin (12) includes a first portion comprising a first analyte-specific sensor (220) for sensing a first analyte in stimulated sweat and a sweat stimulating component (240); and a second portion comprising a second analyte-specific sensor (225, 226) for sensing a second analyte in natural sweat.

### BACKGROUND

Sweat sensing technologies have enormous potential for applications ranging from athletics, to neonatology, to pharmacological monitoring, to personal digital health, to name a few applications. Sweat contains many of the same biomarkers, chemicals, or solutes that are carried in blood and can provide significant information enabling one to diagnose ailments, health status, toxins, performance, and other physiological attributes even in advance of any physical sign. Furthermore, sweat itself, the action of sweating, and other parameters, attributes, solutes, or features on, near, or beneath the skin can be measured to further reveal physiological information. However, obtaining a sweat sample free of contamination is challenging.

Biosensing using sweat has many drawbacks and limitations that must be resolved in a manner that affordably, effectively, conveniently, intelligently, and reliably brings sweat sensing technology into intimate proximity with sweat as it is generated.

### SUMMARY OF THE INVENTION

Embodiments of the disclosed invention provide a device for sensing sweat on skin. In an embodiment, a device includes a first analyte-specific sensor for sensing a first analyte in sweat; a sweat stimulating component; a waste collector beneath the sweat stimulating component; and an additional component being one or more of a sweat collector or an additional analyte-specific sensor for sensing an additional analyte in sweat that is different from the first analyte, the additional component being adjacent to and fluidically isolated from the waste collector.

In another embodiment, a device for sensing sweat on skin includes a first portion comprising a first analyte-specific sensor for sensing a first analyte in stimulated sweat and a sweat stimulating component; and a second portion comprising a second analyte-specific sensor for sensing a second analyte in natural sweat.

The objects and advantages of the disclosed invention will be further appreciated in light of the following detailed descriptions and drawings.

### BRIEF DESCRIPTION OF THE DRAWINGS

FIG. 1A is a cross-sectional view of a sweat sensing device according to an embodiment of the disclosed invention.

FIG. 1B is a cross-sectional view partially broken away of the device taken along the line 1B-1B of FIG. 1A.

FIG. 1C is a top view of the device of FIG. 1B with some components removed for the sake of clarity.

FIG. 2 is a cross-sectional view of a sweat sensing device according to another embodiment of the disclosed invention.

### DETAILED DESCRIPTION OF THE INVENTION

Reference throughout this specification to “one embodiment” or “an embodiment” means that a particular feature, structure, material, or characteristic described in connection with the embodiment is included in at least one embodiment of the invention, but does not denote that they are present in every embodiment. Thus, the appearances of the phrases “in an embodiment” or “in another embodiment” in various places throughout this specification are not necessarily referring to the same embodiment of the invention. Further, “a component” may be representative of one or more components and, thus, may be used herein to mean “at least one.”

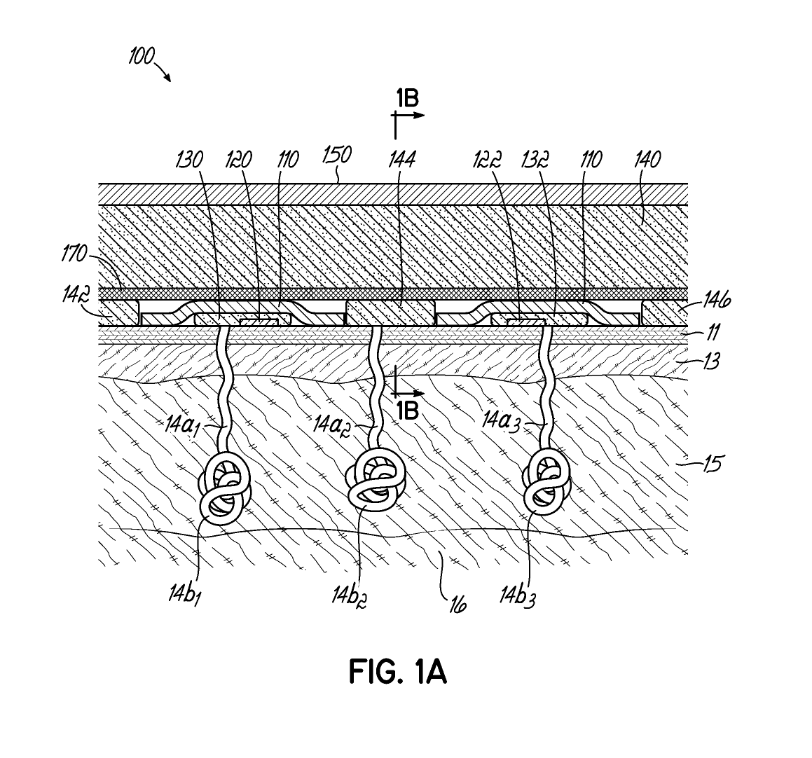

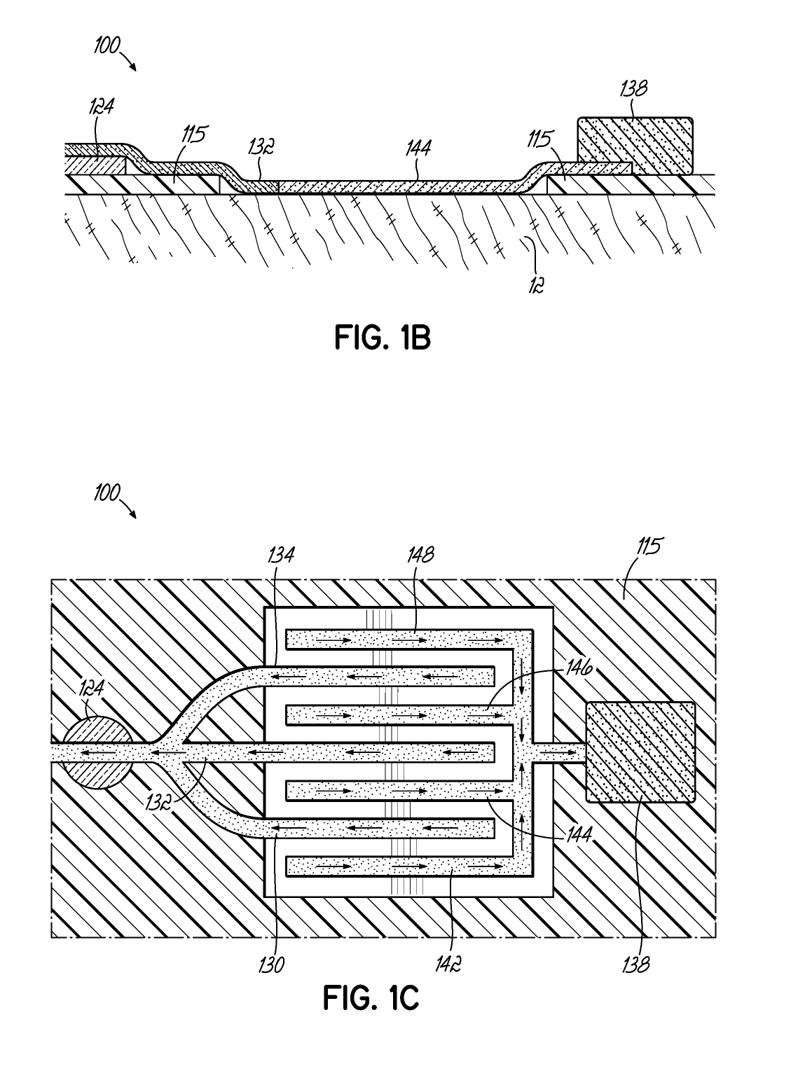

With reference to FIGS. 1A-1C, a sweat sensing device 100 according to an embodiment of the disclosed invention is shown. The device 100 is positioned on skin 12 composed of the stratum corneum 11, the epidermis 13, the dermis 15, and layers of skin below the dermis 16. The skin 12 contains multiple sweat glands, each having a ductal lumen 14a1, 14a2, 14a3 and secretory coil 14b1, 14b2, 14b3. The device 100 is capable of indirect and/or direct sweat stimulation and includes at least one sensor specific to at least one analyte in sweat (e.g., at least one of sensors 120, 122, 124 described below). As described further below, the device includes a collection/sensing area that is fluidically isolated from the stimulation area. The collection/sensing area of the device 100 may comprise one or more sweat collectors 130, 132 and optionally one or more sensors 120, 122. In the illustrated embodiment, the sensors 120, 122 are contained inside a corresponding sweat collector 130, 132 where the sweat collectors 130, 132, 134 contact the skin 12. In another embodiment, the sensors 120, 122 may be located beneath a corresponding sweat collector 130, 132, 134 (i.e., on the skin 12 or between the sweat collector and the skin 12 if there is an intervening layer).

Still referring to FIGS. 1A-1C, the device 100 further comprises a stimulant reservoir, such as a stimulant gel 140, a series of waste collectors 142, 144, 146, 148, a sweat impermeable adhesive 110, and a waste reservoir 138. The waste reservoir 138 may be a wicking component such as a wicking pump. The waste collectors 142, 144, 146, 148 serve three purposes: (1) they allow proper contact with the surface of the skin 12 for iontophoresis; (2) they allow the stimulant to pass through to the skin 12; and (3) they transport sweat generated between them and the surface of the skin 12 away from the skin 12. The sweat impermeable adhesive 110 serves a dual function of holding components of the device 100 together and separating the stimulation area (e.g., the area adjacent the waste collectors 142, 144, 146, 148) from the collection area (e.g., the area adjacent the sweat collectors 130, 132, 134) to prevent cross contamination. In other words, the sweat impermeable adhesive 110 fluidically isolates the sweat collectors 130, 132, 134 and the waste collectors 142, 144, 146, 148. The waste reservoir 138 is coupled to the waste collectors 142, 144, 146, 148 and collects excess waste fluid such as sweat. The wicking waste reservoir 138 has a wicking or capillary pressure that is less than that of the stimulant gel 140 such that it does not pull fluid from the stimulant gel 140. In an embodiment, the stimulant reservoir could also be a hydrogel, which may lose some water due to contact with the wicking waste reservoir 138, but not all, because hydrogels also have a strong wicking capacity. Alternately, the stimulant reservoir could be a volume-stable reservoir. In an embodiment, the volume-stable stimulant reservoir includes a gel or solution that is held within a rigid reservoir, such that its fluidic volume does not change due to an increase in sweat or due to the wicking influence of the waste reservoir 138. For example, the stimulant reservoir could be a rigid porous material (e.g., a porous ceramic) filled with an aqueous solution of sweat stimulant, a sealed polymer, or a metal vessel. In another embodiment, a volume-stable stimulant reservoir has an osmolality similar to sweat, such that osmotic pressure ensures the stimulant reservoir does not gain nor lose too much water.

With further reference to FIG. 1A, the device 100 further includes an iontophoresis electrode 150 and a membrane 170. Suitable materials for the membrane 170 include a track-etch membrane, a dialysis membrane, a forward osmosis membrane, or other suitable membrane capable of maintaining the integrity of the stimulant gel 140 even in the presence of sweat in the waste collectors 142, 144, 146, 148. For example, typically with iontophoresis less than 5% of the total chemical stimulant can be delivered into the skin 12 during use of a device such as device 100, and the membrane 170 therefore could retain a chemical in the stimulant gel 140 such that at least 10% or at least 50% of the chemical would remain in the stimulant gel 140 even after at least 24 hours of use of the device 100. The stimulant reservoir, such as a stimulant gel 140, may include a slowly metabolizing chemical stimulant such as carbachol or a conventional stimulant such as pilocarpine. The membrane 170 can also separate the stimulant gel 140 from the skin 12 and, while slowing flow of sweat into the stimulant gel 140, allows iontophoresis of stimulant through the membrane 170. In an embodiment, the membrane 170 has a hydrophilic side facing the stimulant gel 140 and a hydrophobic side facing the other direction. The stimulant is iontophoretically delivered from the stimulant gel 140, through the membrane 170, through the waste collectors 142, 144, 146, 148, and into the skin using the iontophoresis electrode 150. Because the spacing between adjacent waste collectors 142, 144, 146 is small enough (mm's or smaller), stimulant causes sweat to be generated at all glands that can be activated including those areas in between waste collectors 142, 144, 146, 148. The stimulant could be driven to near secretory coils of glands, such as 14b1 and 14b3, by current spreading or divergence of the electric field driving the iontophoresis or by diffusion through the dermis. Alternately, the stimulant could cause indirect stimulation of sweat using sudo-motor-axon reflex sweating (e.g. stimulation of gland 14b2 and a coupled sweat response via the nerve axons to glands 14b1 and 14b3. The stimulant does not pass through the sweat impermeable adhesive 110 even during iontophoresis.

The terms sweat collectors and waste collectors as used herein are not limited to wicking components. Sweat and waste collectors may broadly include any element capable of transporting sweat away from the skin and into the device. Suitable materials include gels, textiles, microfluidic channels or cavities, tubes, or any other suitable element. In an embodiment, the waste collectors 142, 144, 146, 148 are made of a woven or non-woven textile (e.g., polyester) that is embedded with a gel (e.g., agar) where the textile component provides structural support during assembly of the device. Alternately, the waste collectors 142, 144, 146, 148 are made of a textile (e.g., Rayon) with no gel, and the fibers of the textile are kept wet by fluid coming from stimulant gel 140. Therefore, when wet, the textile provides an electrically conductive pathway for iontophoresis delivery of the sweat stimulant into skin. Excess sweat generated could then fill the larger pores inside a Rayon textile and be transported away as needed. Although the illustrated embodiment includes three sweat collectors and four waste collectors, it should be recognized that the number of sweat collectors and/or waste collectors may vary. Each of the sweat and waste collectors may cover areas ranging, for example, from several mm² to cm² or more.

With reference to FIGS. 1B and 1C, in which the membrane 170, stimulant gel 140, and iontophoresis electrode 150 are not shown for clarity, the device 100 includes an analyte-specific sensor 124 and a sweat impermeable substrate 115, which may be an adhesive. To sense sweat, embodiments of the disclosed invention include at least one sensor specific to at least one analyte in sweat. Therefore, in an embodiment where sensors 120, 122 are not present, one or more analyte-specific sensors 124 are included; in an embodiment including at least one of sensors 120, 122, the sensor 124 is optional. Relative to the sensor 124, the sensors 120, 122 are closer to the skin 12. In an embodiment including two or more sensors, the sensors may be configured to sense different analytes. For example, in an embodiment with sensors 120, 122, 124, sensors 120, 122 could be ion-selective electrodes to sense Na+ and K+, respectively, and are used to quickly measure sweat generation rate and therefore predict the sampling interval or rate for the device 100 (Na+ increases with sweat generation rate, K+ does not and is a baseline). The sensor 124 could then be an electrochemical aptamer sensor specific to, for example, cortisol. Where the sensor 124 would have potential issues with abrasion and/or electrical noise, it is spaced apart from the surface of the skin 12.

With reference to FIG. 1C, in which the sweat impermeable adhesive 110 is not shown for clarity, the spacing between the four waste collectors 142, 144, 146, 148 and the three sweat collectors 130, 132, 134 is illustrated. Each of the waste collectors 142, 144, 146, 148 and the sweat collectors 130, 132, 134 may have equal or different wicking pressures so long as individually each wicking element serves its purpose. The sweat collectors 130, 132, 134 wick sweat from the skin 12 to the sensor 124. The sweat collectors 130, 132, 134 could also lead to a waste reservoir similar to the waste reservoir 138 (not shown) after the sweat is sensed. Alternately, with proper design of wicking pressures, both the sweat collectors 130, 132, 134 and the waste collectors 142, 144, 146, 148 could all share use of a common waste reservoir (not shown). In addition, alternate arrangements of sweat and waste collectors are possible, and the specific geometries, sizing, and spacing shown herein for the device 100 are just one possible example. The configuration of the fluidically isolated sweat and waste collectors may vary as long as the configuration allows the stimulant to pass through the waste collectors to directly or indirectly generate sweat under the sweat collectors, which are adjacent to (i.e., side-by-side) the waste collectors, and the sweat and waste collectors transport sweat away from the skin.

It is understood that sweat can also emerge from the skin 12 underneath the adhesive 110. The adhesive 110 can still provide effective isolation between waste collectors 142, 144, 146, 148 and sweat collectors 130, 132, 134 even if sweat emerges beneath the adhesive 110. For example, if the adhesive 110 simply reduces the fluidic volume underneath the adhesive 110, sweat will experience positive pressure and be pushed to the edges of the adhesive 110 where it is then collected by the waste collectors 142, 144, 146, 148 and sweat collectors 130, 132, 134. Simply, the positive pressure of sweat generation provides an advective flow that significantly fluidically isolates waste collectors 142, 144, 146, 148 and sweat collectors 130, 132, 134. Therefore, the adhesive 110 can be any material (adhesive, silicone elastomer, plastic coated with grease, etc.) that restricts fluid flow between waste collectors 142, 144, 146, 148 and sweat collectors 130, 132, 134. For example, adhesive 110 could be used to prevent less than 30%, less than 10%, less than 5%, or even less than 1% by volume mixing of fluid between waste collectors 142, 144, 146, 148 and sweat collectors 130, 132, 134.

A method of making a device according to an embodiment of the disclosed invention is now described. For illustrative purposes, the reference numbers used to describe the components of the device 100 are used. First, the stimulant gel 140 and the electrode 150 are placed in close contact with little to no space therebetween to allow for the proper flow of electric current into the stimulant gel 140 allowing the charged stimulant to be carried into the body. Next, the membrane 170 is placed down on the stimulant gel 140 with a hydrophilic side facing the stimulant gel 140 and a hydrophobic side facing the other direction. Note, it is possible to treat the membrane 170 with different coatings to promote more or less ion flow. This is designed to allow mainly a single directional flow of stimulant into the body and reduce the cross contamination of the stimulant gel 140 once sweating occurs. Additionally, the hydrophobic side allows for the sweat impermeable adhesive 110 to stick to the membrane 170. At this point, the electrode 150, stimulant gel 140, and membrane 170 are upside down (i.e., the layers are in reverse order compared to when the completed device is placed on skin 12) and form a first part. In a separate step, a collection wicking layer 130, 132, 134 (wicks sweat away from the collection area to a sensor), an adhesive 110, and a stimulation wicking layer 142, 144, 146, 148 (allows for the passing of stimulant to the skin 12 and wicks away excess sweat from the stimulation area) are all aligned and laminated together to form a second part. A carrier may be included in the second part to prevent unintended adhesion by the adhesive 110. The carrier could be, for example, wax or siliconized paper such as that used in bandage backings. The first and second parts are then laminated creating the final device. Once the carrier is removed, the device may be placed on the skin 12.

In use, the electrode 150 of the device 100 is used to iontophoretically deliver the stimulant in the stimulant gel 140 to the skin 12. The stimulant moves from the stimulant gel 140, through the waste collectors 142, 144, 146, 148, and into the skin 12. The area on the skin 12 through which the stimulant passes is the stimulation area. The adhesives 110, 115 act to substantially block the stimulant from reaching the sensors 120, 122 and/or the sweat collectors 130, 132, 134. The area of the skin 12 adjacent the sensors 120, 122 and/or the sweat collectors 130, 132, 134 is the collection area. After the stimulant causes sweating, the sweat reaches the sensors 120, 122 and/or the sweat collectors 130, 132, 134 and the waste collectors 142, 144, 146, 148. Thus, sweat is generated in both the stimulation area and the collection area. In an embodiment where the sensors 120, 122 are present, the sensors 120, 122 sense the generated sweat. In an embodiment where the sweat collectors 130, 132, 134 are present, they wick sweat away from the skin 12. If the optional sensor 124 is present, the sweat collectors 130, 132, 134 wick sweat to the sensor 124. The waste collectors 142, 144, 146, 148 wick excess sweat to the waste reservoir 138. In this manner, the configuration of the device 100 separates the sweat that reaches the sensors 120, 122, 124 from the area used to stimulate the sweat. In other words, the potential for contamination of the sensed sweat with the stimulant is significantly reduced, and vice-versa.

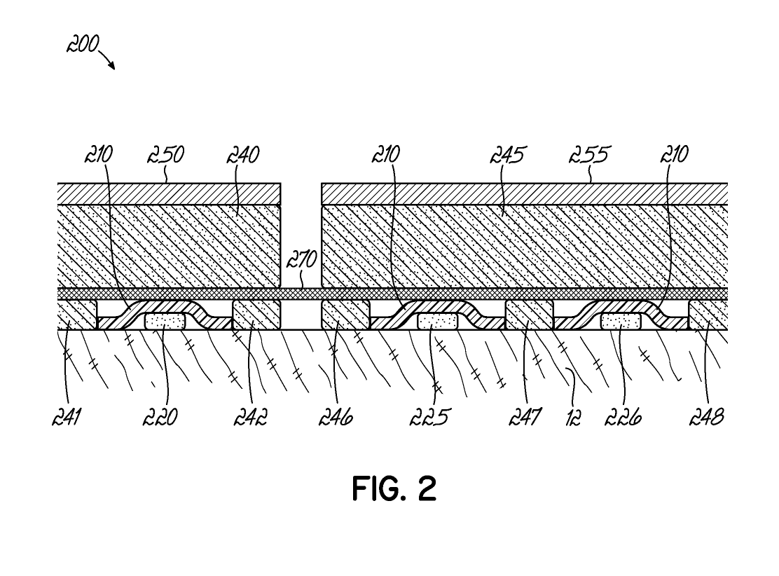

With reference to FIG. 2, in an embodiment of the disclosed invention, a device 200 is positioned on the skin 12 and includes an adhesive 210, a membrane 270, and two separate portions each capable of sweat stimulation, collection, and sensing. For example, the waste collectors 241, 242 and sensors or sweat collector 220, along with stimulant gel 240 and electrode 250, collectively form a first portion of the device 200 for stimulation of a first sweat generation rate and sensing of at least a first analyte in sweat. The waste collectors 246, 247, 248 and sensors or sweat collectors 225, 226, along with stimulant gel 245 and electrode 255, collectively form a second portion of the device 200 for stimulation of a second sweat generation rate and sensing of at least a second analyte in sweat. For example, the first sweat generation rate could be a high rate (e.g., 1 nL/min/gland), and the first portion may be used for sensing cortisol and dehydroepiandrosterone in sweat with sampling intervals of 5 minutes. The second sweat generation rate could be 10× lower (e.g., 0.1 nL/min/gland), and the second portion may be used to measure vasopressin, which could be diluted at higher sweat rates and therefore benefit from being sensed from sweat that is secreted at low sweat generation rates. While two separate portions are shown, it should be recognized that more than two separate portions may be included in another embodiment.

With reference to FIG. 2, in another embodiment, one of the portions of the device 200 may be used to sense naturally generated sweat, while the other of the portions is used to sense stimulated sweat. Thus, one of the portions may not perform sweat stimulation and the stimulation components may be excluded for that portion, while the other portion performs sweat stimulation and therefore includes the stimulation components. In an embodiment, the first portion could measure natural sweating and Na+ (or another measure of sweat rate) to measure water loss and the potential for dehydration if the user does not take in water, and the second portion could measure vasopressin and/or urea using stimulated sweat and, therefore, directly measure for dehydration. Such a multi-portion embodiment could also be useful for measuring any analyte(s) that are confounded by sweat stimulation. For example, sweat stimulation confounds measurement of analytes for predicting natural sweat rate, such as Na+. The analyte-specific sensors in each portion may sense the same or different analytes.

One skilled in the art will recognize that the various embodiments may be practiced without one or more of the specific details described herein, or with other replacement and/or additional methods, materials, or components. In other instances, well-known structures, materials, or operations are not shown or described in detail herein to avoid obscuring aspects of various embodiments of the invention. Similarly, for purposes of explanation, specific numbers, materials, and configurations are set forth herein in order to provide a thorough understanding of the invention. Furthermore, it is understood that the various embodiments shown in the figures are illustrative representations and are not necessarily drawn to scale.

While specific embodiments have been described in considerable detail to illustrate the disclosed invention, the description is not intended to restrict or in any way limit the scope of the appended claims to such detail. The various features discussed herein may be used alone or in any combination. Additional advantages and modifications will readily appear to those skilled in the art. The invention in its broader aspects is therefore not limited to the specific details, representative apparatus and methods and illustrative examples shown and described. Accordingly, departures may be made from such details without departing from the scope of the general inventive concept.

What is claimed is:

1. A device for sensing sweat on skin comprising:
a first analyte-specific sensor for sensing a first analyte in sweat;
a sweat stimulating component;
a waste collector between the sweat stimulating component and the skin; and
a first additional component being one or more of a sweat collector or an additional analyte-specific sensor for sensing an additional analyte in sweat that is different from the first analyte, the first additional component being adjacent to and fluidically isolated from the waste collector.

2. The device of claim 1, wherein the first additional component is the sweat collector and the waste collector and the sweat collector wick sweat to different locations.

3. The device of claim 2, wherein the sweat collector wicks sweat to the first analyte-specific sensor.

4. The device of claim 1, further comprising:
a sweat reservoir fluidically coupled to the waste collector, wherein the waste collector wicks excess sweat to the sweat reservoir.

5. The device of claim 1, wherein the additional component is the sweat collector, the device further comprising:
a sweat reservoir fluidically coupled to the sweat collector, wherein the sweat collector wicks sensed sweat to the sweat reservoir

6. The device of claim 5, wherein the waste collector wicks excess sweat to the sweat reservoir.

7. The device of claim 1, wherein the first additional component is the additional analyte-specific sensor, and wherein the additional analyte-specific sensor is for determining a sweat rate.

8. The device of claim 1, further comprising:
a sweat impermeable material for fluidically isolating the waste collector and the first additional component.

9. The device of claim 7 wherein the sweat impermeable material is an adhesive.

10. The device of claim 1, further comprising:
a stimulating component reservoir that contains the sweat stimulating component; and
an iontophoresis electrode for driving the sweat stimulating component into skin.

11. The device of claim 9, wherein said stimulating component reservoir is volume-stable.

12. The device of claim 1, further comprising:
at least one membrane, wherein said sweat stimulating component and said waste collector are separated by said at least one membrane.

13. The device of claim 11 wherein said sweat stimulating component contains at least one chemical to stimulate sweat and said membrane causes said sweat stimulating component to retain at least one of 10% or 50% of the original concentration of said chemical over a period of at least 24 hours.

14. The device of claim 1, further comprising:
a first portion comprising the first analyte-specific sensor and the first additional component, the first portion for sensing a first analyte at a first sweat generation rate; and
a second portion comprising a second analyte-specific sensor and a second additional component, the second portion for sensing a second analyte different from the first analyte at a second sweat generation rate.

15. A method for sensing sweat on skin comprising:
directing a sweat stimulant into a stimulation area of the skin;
transporting stimulated sweat from the stimulation area away from the skin; and
sensing stimulated sweat from a collection area of the skin, the collection area of the skin being fluidically isolated from the stimulation area of the skin.

16. The method of claim 14, wherein sensing stimulated sweat includes sensing a first analyte in sweat using a first analyte-specific sensor.

17. The method of claim 15, further comprising:
transporting stimulated sweat from the collection area of the skin towards the first analyte-specific sensor.

18. The method of claim 14, wherein transporting stimulated sweat from the stimulation area away from the skin includes transporting the stimulated sweat to a sweat reservoir.

19. The method of claim 14, wherein the collection area is a stimulated sweat collection area, the method further comprising:
sensing natural sweat from a natural sweat collection area separate from the stimulated sweat collection area.

20. The method of claim 18, wherein sensing natural sweat includes sensing a natural sweat rate.

21. A device for sensing sweat on skin comprising:
a first portion comprising a first analyte-specific sensor for sensing a first analyte in stimulated sweat and a sweat stimulating component; and
a second portion comprising a second analyte-specific sensor for sensing the first analyte or a second analyte in natural sweat.

22. The device of claim 20, wherein the first portion is configured to sense a biomarker for dehydration and the second portion is configured to sense a natural sweat rate.

23. The device of claim 21, wherein the biomarker for dehydration is at least one of urea or vasopressin.

24. The device of claim 20, wherein the first portion further comprises a waste collector between the sweat stimulating component and the skin and an additional component being one or more of a sweat collector or an additional analyte-specific sensor, the additional component being adjacent to and fluidically isolated from the waste collector.
